(19) United States
(12) Patent Application Publication
(10) Pub. No.: US 2002/0015988 A1
(43) Pub. Date: Feb. 7, 2002

(54) Granular formulation containing microorganisms, a process for the preparation and the use thereof

(76) Inventors: Margarete Enzmann, Weil am Rhein (DE); William Baettig, Pratteln (CH)

Correspondence Address: William A. Teoli, Jr., Syngenta Crop Protection, Inc., Patent and Trademark Dept., 410 Swing Road, Greensboro, NC, 27409, US

(21) Appl. No.: 09/785,821

(22) Filed: Feb. 16, 2001

Related U.S. Application Data
(63) Continuation of application No. 09/207,374, filed on Dec. 8, 1998, now abandoned.

(30) Foreign Application Priority Data
Jul. 3, 1995 (EP) PCT/EP95/02571
Jul. 14, 1994 (CH) 2254/94

(51) Int. Cl. C12N 1/20

(52) U.S. Cl. 435/252.1

(57) ABSTRACT

The invention relates to a) a film-forming, water-soluble and essentially uncrosslinked polymer, and the granular formulation contains not less than 0.5% by weight of water, based on said formulation, or

b) a film-forming, structurally crosslinked polysaccharide which contains carboxyl or sulfate groups and is swellable in water in the presence of potassium ions, and the granular formulation contains not less than 0.5 % by weight of water, based on said formulation. The invention further relates to a process for the preparation of said granular formulation and to the use thereof for protecting plants from attack by disease or damage by insects.

The present invention relates to a granular formulation comprising (1) a solid water-insoluble and finely particulate substrate, (2) a water-soluble or water-swellable film-forming polymer which is not covalently crosslinked or which is crosslinked with polyvalent cations, (3) microorganisms and (4) water. The invention further relates to a process for the preparation of said granular formulation and to the use thereof for protecting plants against diseases and attack by insects.

Plant protection using spore-forming or vegetative cells (microorganisms) has recently attained increasing importance. A prerequisite for the use of such biological control agents is the ability to process them to useful formulations such as suspension concentrates, dispersible powders, granules or, in particular, scattering granules. The preparation of formulations is, however, fraught with great difficulties. For example, no process for the preparation of most vegetative cells, and also for some spores, can be used that utilises temperatures higher than c. 40° C., as the microorganisms are thereby damaged and a substantial loss in viability is observed. Storage likewise poses a problem, as it is not possible to avoid losses in viability under ambient conditions resulting from cell death or when it is necessary to store the formulations at low temperature to avoid loss of viability.

Most known formulations of microorganisms consist of polymer gels crosslinked with polyvalent cations containing these microorganisms. Such a formulation is described, inter alia, by D. R. Fravel et al. in Phytopathology, Vol. 75, No. 7, 774-777, 1985, using alginate as polymer material. The concurrent use of substrates is disclosed therein. The preparation of these formulations is usually effected by mixing solutions of natural or synthetic gel-forming polymers, for example alginates and aqueous solutions of polyvalent metal ions so as to form individual droplets and such that the microroganisms can be suspended in one of the two, or in both, reaction solutions. The gel formation commences when the suspension of the microorganism is added dropwise to the solution of the gelling agent. These gel particles can be subsequently dried. This process is called ionotropic gelation. Depending on the degree of drying, this process afford compact and hard pellets of polymers that are crosslinked by polyvalent cations and which contain the microorganisms and a substrate in substantially uniform distribution. The particle size can be up to 5 mm.

EP-A1-0 097 571 discloses formulations based on partially crosslinked polysaccharides which, in addition to containing a microorganism, may contain finely particulate silicic acid as substrate and the crosslinking may be effected with Ca⁺⁺ ions. The water activity of the formulations may not be greater than 0.3. In an article reviewing different formulation systems in New Directions in Biological Control: Alternatives for Supressing Agricultural Pests and Diseases, pp. 345-372, Alan R. Liss, Inc. (1990), W. J. Connick et al. refer to granular formulations with vermiculite as substrate and to compact alginate pellets prepared by the ionotropic gelation process. Such formulations are also disclosed by D. R. Fravel in Pesticide Formulations and Application Systems: 11th Volume, ASTM STP 1112 American Society for Testing and Materials, Philadelphia, 1992, pp. 173-179.

These crosslinked gel formulations suffer the drawback of slow release of biological control agent, as the gels are water-insoluble and usually large particles having a diameter greater than several millimetres are formed. If a more rapid release is desired, the formulations have to be pretreated with, typically, buffer solutions. This is more difficult for the end user and limits handling safety. At higher population densities (>10⁹ CFU/g=colony forming units/g), which are necessary for reducing the rate of application, the systems usually do not have sufficient stability and cool storage is necessary to avoid substantial losses. To prepare the formulations, the gel-forming polymers have to be dissolved in water, which is in some cases difficult and only possible at elevated temperature. The dropwise gel formation is a necessary process step to obtain a useful granular formulation. The provision of technical apparatus for carrying out such a process on an industrial scale must be regarded as difficult and expensive. The particles so obtained still have a high water content, which must be reduced by drying to ensure acceptable storage stability. This drying step makes the process even more expensive, subjects the microorganisms to the risk of additional damage and can further diminish their viability. Storage-stable granular formulations based on water-soluble or water-swellable polymers and prepared without ionotropic gelation are not yet known in the art.

Surprisingly, it has now been found that it is possible (a) to prepare granular formulations of microorganisms in a polymer layer without ionotropic gelation and partially without completely dissolving the polymer, (b) to diminish substantially the losses caused by the death of living cells when drying, (c) to achieve a high storage stability, in particular in ambient conditions, (d) to obtain very high population densities of microorganisms and still ensure excellent storage stability, (e) to obtain a rapid release of biological control agent, and (f) to effect excellent stabilisation of particularly vegetative bacteria cells, by applying the microorganisms, in a water-soluble or water-swellable film-forming polymer which is not covalently crosslinked or which is crosslinked by polyvalent cations, to a substrate or together with a substrate, the formulation containing not less than 0.5% by weight of water, based on the entire formulation.

One object of the invention is a granular formulation comprising a finely particulate substrate and a polymer layer containing microorganisms, said polymer being a) a film-forming, water-soluble and essentially uncrosslinked polymer, and the granular formulation contains not less than 0.5% by weight of water, based on said formulation, or b) a film-forming, structurally crosslinked polysaccharide which contains carboxyl or sulfate groups and is swellable in water in the presence of potassium ions, and the granular formulation contains not less than 0.5% by weight of water, based on said formulation.

Essentially uncrosslinked will be understood as meaning in the context of this invention that no monomeric crosslinking agents which lead to the formation of covalent bonds, or no polyvalent cations which result in ionotropic gelation, are added.

Structurally crosslinked means in the present context the formation of a spatial network of a single polymer or of a mixture of two polymers through hydrogen bonds or through the electrostatic interaction of potassium ions. A thermoreversible spatial structure (gel) is thereby achieved which, when heated, goes again into solution. Typical examples are the pronounced double helix structure of carragheenan in the presence of potassium ions or the structural formation of the mixture of carragheenan and locust bean gum. A thermally irreversible structural formation by polyvalent ions does not fall under the above definition.

One or more than one carboxyl or sulfate group may be present per structural repeating unit in the polysaccharide.

Water-soluble means in the present context that it is possible to prepare an least 0.5% by weight aqueous polymer solution in the temperature range from 5 to 95° C.

The granular formulation contains the microroganisms preferably in an amount of 0.1 to 10% by weight, preferably 0.3 to 5% by weight and, most preferably, 0.5 to 3% by weight of dry matter, based on 1 kg of formulation. The sum of all components of the granular formulation is always 100%.

The population density, based on the cell concentration, can be particularly high. The preferred population density of microorganism is from 1×10⁵ bis 1×10¹¹ CFU (colony forming units) per g of granular formulation. During storage at room temperature, this concentration of living cells can be retained in the formulation of this invention over a period of up to 10 months with only minor losses of microorganism of less than one factor of ten CFU.

The residual water content is preferably not less than 1% by weight, more preferably not less than 3% by weight and, most preferably, not less than 5% by weight. The upper limit of the water content is preferably not more than 40% by weight, more preferably not more than 30% by weight and, most preferably, not more than 20% by weight. The upper limit of the water content is governed by the carrier, the water solubility of the polymer and of the process for the preparation of the formulation. In coating methods, for example fluidised bed coating, a water content of 0.5 to 20% by weight is readily achievable, whereas in extrusion methods the water content can be higher and may typically be from 0.5 to 40% by weight.

The finely particulate substrate can have an average particle size of 1 μm to 0.8 cm, more preferably from 10 μm to 0.5 cm and, most preferably, from 20 μm to 0.2 cm. The substrate may be an inorganic or organic material. It is preferred to use organic materials for fungi and inorganic materials for vegetative cells (bacteria). Typical examples of water-insoluble organic materials are comminuted bran, straw, sawdust and cellulose. Particularly suitable inorganic substrates are water-insoluble metal oxides and metal salts (SiO₂, Al₂O₃, BaSO₄, CaCO₃) or silicates and aluminosilicates of alkali metals and alkaline earth metals. Among the silicates, the sheet silicates are preferred. Typical examples of silicates are mineral clays, attapulgite, kieselgur, powdered lime, diatomaceous earth, wollastonite, olivin, montmorillonite and vermiculite. Vermiculite is particularly preferred.

The amount of substrate may typically be from 50 to 99% by weight, preferably from 65 to 95% by weight and, most preferably, from 75 to 90% by weight.

The granular formulation can have an average particle size of 0.01 mm to 8 mm. A preferred average particle size is from 0.2 to 4 mm and a particularly preferred average particle size is from 0.5 to 2 mm.

The film-forming, water-soluble and essentially uncrosslinked polymer can be a synthetic or a natural polymer. Typical examples of synthetic polymers are homo- and copolymers of polyvinyl alcohol, polyethylene glycol or polyvinyl pyrrolidone as well as polyacrylamides. The natural polymers are mainly polysaccharides which may be derivatised. Preferred natural known polymers are legion and are typically starch, alginates, carragheenans, preferably κ-carragheenan, ι-carragheenan, λ-carragheenan, xanthane, locust bean gum or methyl celluloses. Mixtures thereof can also be used.

The polymers must be compatible with the microorganism. Compatibility can be established by those skilled in the art in simple manner by bringing together microorganism and polymer.

Alginates and carraghenans are particularly preferred. A particularly preferred combination of carrier and water-soluble polymer is vermiculite with κ-carraghenan.

The film-forming structurally crosslinked, water-swellable polymer is a polysaccharide, preferably κ-carragheenan, ι-carragheenan, locust bean gum, xanthane, or a mixture thereof, which forms in the presence of potassium ions. These polymers form thermally reversible gels which are distinguished by intermolecular hydrogen bonds or ionic bonds.

The amount of water-soluble or water-swellable polymer may be from 0.1 to 20% by weight, preferably from 0.1 to 10% by weight and, most preferably, from 0.5 to 5% by weight.

The molar ratio of potassium ions to the carboxyl or sulfate groups of the polymer is from 0.001:1 to 1:1.

Microorganisms which can be used for pest control or for controlling plant diseases in agriculture are known and described, inter alia, in EP-A-0 472 494.

Suitable microorganisms are mono- or multicellular fungi or bacteria, typically including Rhizobium spp., Metharizium, Fusarium, Trichoderma, Stryptomyces, Gliocladium, Penicillium, Talaromyces, Verticillium or Colletotrichum. Preferred microorganisms are Pseudomonas spp., Serratia spp., Bacilus spp., Agrobacter spp., Exserohilum spp., Enterobacter spp. The microorganism Pseudomonas aurantiaca ATTC No. 55169 is particularly preferred.

Weeds, insects and fungal diseases which can be controlled with microorganisms are typically Rhizoctonia solani, Rhizoctonia oryzae, Phytium ultimum, Fusarium oxysporum spp., Alphanomyces laevis, Phytophtora infestans, Botryts spp., Sclerotinia sclerotiorum, Bacillus sp., Microdochium nivale, Thielaviopsis basicola, Gaeumanomyces graminis and, in principal, all other diseases caused by pathogenic microorganisms (Erwinia carotovora, Saccaromyces cerevisiae, Xanthomonas vesicatoria, Pseudomonas syringae).

Pseudomonas aurantiaca ATTC No. 55169 is active against a number of the diseases listed above in different crops. The protective action against Rhizoctonia solani in cotton, cucurbits, cabbages, geraniums, impatiens and poinsettia, is particularly marked.

The preparation of classical droplet granular formulations (e.g. Connik W. J.: “Formulation of living biological control agents with alginate” in American Chemical Society, ACS Symposium Series 1988, Vol. 371, pp. 241-250; Fravel D. R, Marois J. J., Lumsden R. D., Connik W. J.: “Encapsulation of potential biocontrol agents in alginate” aus Phytopathology, 1985, Heft 75, S.774-777; Stormo K. E., Crawford R. L. : “Preparation of encpsulated microbial cells for environmental application” in Applied and Environmental Microbiology, 1992, pp. 727-730) gives granules which are virtually insoluble and dissolve only very slowly even in buffer solution, so that a release of the microorganisms takes place very slowly or not at all.

Surprisingly, it has been found that the granules prepared by the process of this invention effect a very rapid release of the microorganisms. The formulation decomposes in buffer or in water, depending on the polymer, over 0.5 to several hours, i.e. the polymer layer becomes detached or swells, so that the entire microbial content is released in the soil within 24 hours.

A further object of the invention is a process for the preparation of a granular formulation comprising a finely particulate substrate and a polymer layer containing microorganisms, said polymer being

a) a film-forming, water-soluble and essentially uncrosslinked polymer, and the granular formulation contains not less than 0.5% by weight of water, based on said formulation, or

b) a film-forming, structurally crosslinked polysaccharide which contains carboxyl or sulfate groups and is swellable in water in the presence of potassium ions, and the granular formulation contains not less than 0.5% by weight of water, based on said formulation, which comprises

(A) to prepare the granules a), suspending or, at a temperature of not higher than 95° C., dissolving, a film-forming and water-soluble polymer and suspending a microorganism in this suspension or solution after cooling to room temperature,

(B) to prepare the granules b), suspending a carboxyl group-containing or sulfate group-containing polysaccharide in an aqueous buffer solution containing potassium ions, and then suspending the microorganism in this solution,

(C) spraying the resultant suspensions direct on to a finely particulate substrate or mixing said suspensions with the finely particulate substrate, and

(D) removing the water to a concentration which is not less than 0.5% by weight, based on the granular formulation.

If a suspension of a film-forming and water-soluble polymer is used for the preparation of granular formulation a), then said suspension is is preferably prepared in the temperature range from 10° to 30° C. To prepare a solution of a film-forming and water-soluble polymer, the temperature range is from 25° to 95° C., depending on the type of polymer.

The addition of the microorganism is made either to the polymer suspension at a temperature below 40° C. or to the cooled polymer solution at a temperature below 40° C., preferably below 30° C.

In another process variant, the granular formulation b) is prepared by dissolving a carboxyl group-containing or sulfate group-containing polysaccharide in an aqueous buffer solution containing potassium ions, at elevated temperature, e.g. 70° C., or by dissolving in identical manner two polymers which interact with each other. A thermally reversible gel forms from these cooled solutions. The addition of the microroganism is made shortly before the solidification point at a temperature below 40° C.

The buffer may be any potassium-containing salt of a polyvalent acid. Commercially available phosphate buffers are particularly preferred Depending on the ratio of dihydrogen phosphate to monohydrogen phosphate, the pH can be adjusted to C. 6.5 to c. 7.5. The preferred pH is 7.

The concentration of buffer is preferably from 0.00001 M/l to 1 M/l, most preferably from 0.005 M/l to 0.05 M/l.

The water is removed under as mild conditions as possible, preferably at room temperature or at slightly elevated temperature up to c. 35° C.

Apparatus for, and methods of, removing the water are known per se. The best method will depend on the viscosity of the batch to be processed. The granular formulations of this invention can be prepared by known methods using conventional apparatus. Spray methods for mixing the components are conveniently used for coating, typically in a fluidised bed reactor. In this method, the solution or suspension of polymer and microorganism is sprayed on to the substrate in the fluidised bed and thereby simultaneously dried.

Another embodiment of the process comprises preparing the novel granular formulations by a known extrusion method. This comprises mixing all components in a mixer with the requisite amount of water and forcing the mixture through a perforated plate. The granules may then be comminuted to the desired size and dried.

Single-screw extruders, granulators, subgranulators, perforated plates and the like may be used.

The process of this invention gives a granular formulation in which the substrate is coated with a thin layer of polymer in which the microorganisms are distributed. What are obtained are usually not discrete coated particles, but agglomerates of a plurality of substrate particles of irregular shape.

Depending on the chosen mixing and drying method, particles of different shape are obtained. Thus the extrusion process gives cylindrical pellets in which substrate and microroganism are coated with polymer material substantially independently of each other, whereas the spray method in the fluidised bed gives agglomerates of substrate in which the particles are coated with a thin polymer layer containing the microorganisms. This particle form is preferred, as a particularly rapid release of the microroganism is effected from the thin polymer layer.

The granular formulations of this invention are at all events solid, free-flowing mixtures which can be used direct as scattering granules. They are simple and safe to handle, as they can be filled direct into mechanical devices for field application. The rates of application may be from 1 kg to 20 kg, depending on the type of microorganism.

The granular formulations of this invention can be used for treating plants, parts of plants or the loci of plants (fruit, blossoms, leaves, stalks, tubers, roots, soil) of different crop plants, and the weeds, harmful insects or diseases occurring there can be inhibited or destroyed.

The granular formulations can be applied simultaneously or in succession with further chemical agents to the areas or plants to be treated. Further chemical agents may be fertilisers, micronutrient donors as well as other substances that influence plant growth. Selective herbicides as well as insecticides, fungicides, bactericides, nematicides, molluscicides or mixtures thereof may be used.

The invention further relates to the use of the granular formulations of this invention for protecting plants from infection by disease or from damage by insects; The control is directed to diseases of crop plants and ornamentals in agriculture and in horticulture, especially in cereals, cotton, vegetables, vines, fruit, oil and floral plants. Exemplary of particularly important vegetable crops are cucurbits, cabbages and beans and, as floral plants, poinsietta, geraniums and impatiens.

The invention is illustrated by the following Examples.

### EXAMPLE A1

10×250 ml of Luria Broth, inoculated with Pseudomonas aurantiaca, ATTC No.55169, is centrifuged off after 16 hours of cell growth on a shaker and the pellet is resuspended in 0.01 M phosphate buffer (K₂HPO₄:KH₂PO₄=1:0.78, pH=7 ) to a concentration of 40 ml. 100 ml of phosphate buffer are heated to 70° C. and 0.7 g of κ-carragheenan are added so as to form a 0.7% solution of κ-carragheenan in 0.01 M phosphate buffer. This solution is cooled to just above the the solidification point and mixed with the microorganism suspension.

This mixture is afterwards sprayed in a fluidised bed on to 100 g of vermiculite, giving a granular formulation of the following composition:

16% residual water

1.5% microorganisms, dry matter

81.9% vermiculite

0.6% κ-carragheenan.

The initial concentration is c. 1.1×10¹⁰ CFU/g (colony forming units).

To assess the storage stability, the concentration is determined at suitable intervals. The following data are obtained:

| Storage time in days | CFU/g at 4° C. | CFU/g at RT |
|---|---|---|
| 0 | 1.1 × 10¹⁰ | 1.1 × 10¹⁰ |
| 20 | 1.2 × 10¹⁰ | 1.2 × 10¹⁰ |
| 130 | 1.0 × 10¹⁰ | 9.1 × 10⁹ |
| 317 | 1.6 × 10⁹ | 1.4 × 10⁹ |

### EXAMPLE A2

5 g of κ-carragheenan are stirred with 40 g of 0.01 M phosphate buffer. Then 10 g of cell pellets (30% dry matter) of Pseudomonas aurantiaca, ATTC No. 55169, prepared in a 50 l fermenter, are added. The polymer-microorganism substance is simultaneously mixed with 120 g of vermiculite powder and then extruded. The granules so obtained are dried to the desired water content in the fluidised bed. The granular formulation has the following composition:

18% residual water

1.8% microorganisms, dry matter

77% vermiculite

3.2% κ-carragheenan.

The initial concentration is c. 3.3×10¹⁰ CFU/g (colony forming units).

| Storage time in days | CFU/g bei 4° C. | CFU/ at RT |
|---|---|---|
| 0 | 3.3 × 10¹⁰ | 3.3 × 10¹⁰ |
| 33 | 3.0 × 10¹⁰ | 2.3 × 10¹⁰ |
| 123 | 6.7 × 10⁹ | 1.6 × 10⁹ |
| 174 | 5.9 × 10⁹ | 7.8 × 10⁸ |

### EXAMPLE A3

250 ml of Luria Broth, inoculated with Pseudomonas aurantiaca, ATTC No.55169, are centrifuged off after 16 hours of cell growth on a shaker, and the pellet is resuspended with 0.01 M phosphate buffer according to Example 1 to a concentration of 40 ml.

The microorganism suspension is mixed with 100 ml of 3% sodium alginate solution in 0.01 M phosphate buffer according to Example 2 and sprayed in a fluidised bed on to 100 g of vermiculite.

A granular formulation of the following composition is obtained:

12% residual water

0.5% microorganisms, dry matter

85.5% vermiculite

2.5% sodium alginate

The initial concentration is c. 7.6×10⁸ CFU/g (colony forming units).

| Storage time in days | CFU/g at 4° C. | CFU/g at RT |
|---|---|---|
| 0 | 7.6 × 10⁸ | 7.6 × 10⁸ |
| 20 | 3.3 × 10⁸ | 2.7 × 10⁸ |
| 74 | 3.3 × 10⁸ | 1.6 × 10⁸ |

A spontaneous mutant of Pseudomonas aurantiaca, ATTC No. 55169, was used for Examples A4 A5. The mutant was obtained as follows: Pseudomonas aurantiaca, ATTC No.55169, is plated out on 0.00005% Rifampicin-containing Luria Agar and spontaneously resistant mutants are isolated in known manner and further cultured The Rifampicin-resistant mutants so obtained are used for the following experiments A4 and A5.

### EXAMPLE A4

250 ml of Luria Broth, inoculated with Pseudomonas aurantiaca, ATTC No. 55169 (Rifampicin-resistant), are centrifuged off after 16 hours of cell growth on a shaker, and the pellet is resuspended with 0.01 M phosphate buffer to a concentration of 42 g according to Example 1. The microorganism suspension is mixed with the same amount of a solution of polyvinyl alcohol (Mowiol 40-88, 16%) and sprayed in a fluidised bed on to 100 g of vermiculite.

A granular formulation of the following composition is obtained:

10% residual water

0.5% microorganisms, dry matter

84% vermiculite

5.5% polyvinyl alcohol

The initial concentration is c. 1.1×10⁹ CFU/g (colony forming units).

| Storage time in days | CFU/g at 4° C. | CFU/g at RT |
|---|---|---|
| 0 | 1.1 × 10⁹ | 1.1 × 10⁹ |
| 70 | 8.3 × 10⁸ | 1.1 × 10⁸ |
| 120 | 7.0 × 10⁸ | 5.3 × 10⁸ |

### EXAMPLE A5

250 ml of Luria Broth, inoculated with Pseudomonas aurantiaca, ATTC No. 55169 (Rifampicin-resistant), are centrifuged off after 16 hours of cell growth on a shaker, and the pellet is resuspended with 0.01 M phosphate buffer to a concentration of 40 ml according to Example 1. The microorganism suspension is mixed with 100 ml of a 3% suspension of κ-carragheenan in 0.01 M phosphate buffer according to Example 2 and sprayed on to 100 g of vermiculite.

A granular formulation of the following composition is obtained:

12% residual water

0.5% microorganisms, dry matter

85% vermiculite

2.5% κ-carragheenan.

The initial concentration is c. 1.1×10⁹ CFU/g (colony forming units).

| Storage time in days | CFU/g at 4° C. | CFU/g at RT |
|---|---|---|
| 0 | 1.1 × 10⁹ | 1.1 × 10⁹ |
| 90 | 3.1 × 10⁸ | 1.4 × 10⁸ |
| 211 | 5.3 × 10⁸ | 5.2 × 10⁸ |

### EXAMPLE A6

8 g of κ-carragheenan are stirred with 40 ml of 0.01 M phosphate buffer. Then 5 g of centrifuged spores of Fusarium nygamai, fermented in a 50 l fermenter on Richard's medium for 120 hours, are added. The polymer-microorganism mixture is mixed uniformly with 120 g of vermiculite powder and then extruded. The granular formulation so obtained is dried in a fluidised bed to the desired water content.

A formulation of the following composition is obtained:

13% residual water

0.5% microorganisms, dry matter

81% vermiculite

5.5% ι-carragheenan.

| Storage time in days | CFU/g at 4° C. | CFU/g at RT |
|---|---|---|
| 0 | 3.8 × 10⁸ | 3.8 × 10⁸ |
| 43 | 2.4 × 10⁸ | 2.8 × 10⁸ |
| 76 | 3.0 × 10⁸ | 1.5 × 10⁸ |
| 119 | 4.2 × 10⁸ | |
| 167 | 1.3 × 10⁸ | 1.4 × 10⁸ |
| 210 | 1.3 × 10⁸ | 1.6 × 10⁸ |

### EXAMPLE B1

### Biocontrol

The granular formulation prepared in Example A1 is tested for its biological activity after specific storage times at room temperature under greenhouse conditions. The standardised test conditions are:

crop plant: cotton

pathogen: Rhizoctonia solani.

The granular formulation is added to the pot substrate in an amount of 16 g/litre of pot substrate.

No loss of biological activity is found after storage for 10 months at room temperature.

What is claimed is:

1. A granular formulation comprising a finely particulate substrate and a polymer layer containing microorganisms, said polymer being
a) a film-forming, water-soluble and essentially uncrosslinked polymer, and the granular formulation contains not less than 0.5% by weight of water, based on said formulation, or
b) a filming, structurally crosslinked polysaccharide which contains carboxyl or sulfate groups and is swellable in water in the presence of potassium ions, and the granular formulation contains not less than 0.5% by weight of water, based on said formulation.

2. A granular formulation according to claim 1, which contains the microorganisms in an amount of 0.1 to 10% by weight, based on 1 kg of said formulation.

3. A granular formulation according to claim 1, which contains the microorganisms in an amount of 0.3 to 5% by weight, based on 1 kg of said formulation.

4. A granular formulation according to claim 1, which contains the microorganisms in an amount of 0.5 to 3% by weight, based on 1 kg of said formulation.

5. A granular formulation according to claim 1, which contains the microorganism in a population density of 1×10⁵ to 1×10¹¹ CFU (colony forming units) per g of said formulation.

6. A granular formulation according to claim 1, wherein the residual water content not less than 1% by weight, based on said formulation.

7. A granular formulation according to claim 1, wherein the residual water content is not less than 3% by weight, based on said formulation.

8. A granular formulation according to claim 1, wherein the residual water content is not less than 3% by weight, based on said formulation.

9. A granular formulation according to claim 1, wherein the maximum water content is not greater than 40% by weight, based on said formulation.

10. A granular formulation according to claim 1, wherein the finely particulate substrate has an average particle size of 1 μm to 0.8 cm.

11. A granular formulation according to claim 1, wherein the finely particulate substrate has an average particle size of 10 μm to 0.5 cm.

12. A granular formulation according to claim 1, wherein the finely particulate substrate has an age particle size of 20 μm to 0.2 cm.

13. A granular formulation according to claim 1, wherein the water-insoluble substrate is an inorganic or organic material.

14. A granular formulation according to claim 13, wherein the water-insoluble substrate is comminuted bran, straw, sawdust or cellulose.

15. A granular formulation according to claim 13, wherein the inorganic substrate is a water-insoluble metal oxide, a metal salt (SiO₂, Al₂O₃, BaSO₄, CaCO₃), a silicate or an aluminosilicate of alkali metals and alkaline earth metals.

16. A granular formulation according to claim 15, wherein the water-insoluble substrate is a mineral clay, attapulgite, kieselgur, powdered lime, diatomaceous earth, wollastonite, olivin, montmorillonite or vermiculite.

17. A granular formulation according to claim 15, wherein the water-insoluable substrate is vermiculite.

18. A granular formulation according to claim 1, wherein the amount of substrate is 50 to 99% by weight, based on said formulation.

19. A granular formulation according to claim 18, wherein the amount of substrate is 65 to 95% by weight, based on said formulation.

20. A granular formulation according to claim 1, wherein the amount of substrate is 75 to 90% by weight, based on said formulation.

21. A granular formulation according to claim 1, which has an average particle size of 0.01 to 8 mm.

22. A granular formulation according to claim 21, which has an average particle size of 0.2 to 4 mm.

23. A granular formulation according to claim 21, which has an average particle size of 0.5 to 2 mm.

24. A granular formulation according to claim 1, wherein the film-forming, water-soluble and essentially uncrosslinked polymer is a synthetic or natural polymer.

25. A granular formulation according to claim 1, wherein the film-forming water-soluble and essentially uncrosslinked polymer is a homo- or copolymer of polyvinyl alcohol, polyethylene glycol or polyvinyl pyrrolidone as well as polyacrylamides.

26. A granular formulation according to claim 1, wherein the film-forming, water-soluble and essentially uncrosslinked polymer is a polysaccharide or derivatised polysaccharide.

27. A granular formulation according to claim 26, wherein the film-forming, water-soluble and essentially uncrosslinked polymer is a starch, alginate, carragheenan, κ-carragheenan, ι-carraghenan, xanthane, locust bean gum, or methyl cellulose, or a mixture thereof.

28. A granular formulation according to claim 27, wherein the film-forming, water-soluble and essentially uncrosslinked polymer is κ-carragheenan, ι-carragheenan or an alginate.

29. A granular formulation according to claim 1, wherein the film-forming, structurally crosslinked, water-swellable, carboxyl group-containing or sulfate group- containing polymer is κ-carragheenan, ι-carragheenan, xanthane, or a mixture of locust bean gum and xanthane.

30. A granular formulation according to claim 1, wherein the film-forming, structurally crosslinked, water-swellable, carboxyl group-containing or sulfate group-containing polymer is κ-carragheenan or ι-carragheenan.

31. A granular formulation according to claim 1, which contains the water-soluble or water-swellable polymer in an amount of 0.1 to 20% by weight, based on said formulation.

32. A granular formulation according to claim 1, wherein the molar ratio of the potassium ions to the carboxyl groups or sulfate groups of the polymer is from 0.001:1 to 1:1.

33. A granular formulation according to claim 1, wherein the microorganism is selected from the group consisting of Rhizobium spp., Metharizium, Fusarium, Trichoderma, Stryptomyces, Gliocladium, Penicillium, Talaromyces, Verticillium oder Colletotrichum, Pseudomonas spp., Serratia spp., Exserohilum spp., Bacilus spp., Agrobacter spp., Enterobacter spp. and Pseudomonas aurantiaca ATTC No. 55169.

34. A granular formulation according to claim 1, wherein the microorganism is Pseudomonas aurantiaca, ATTC No. 55169.

35. A process for the preparation of a granular formulation comprising a finely particulate substrate and a polymer layer containing microorganisms, said polymer being
a) a film-forming, water-soluble and essentially uncrosslinked polymer, and the granular formulation contains not less than 0.5% by weight of water, based on said formulation, or
b) a film-forming, structurally crosslinked polysaccharide which contains carboxyl or sulfate groups and is swellable in water in the presence of potassium ions, and the granular formulation contains not less than 0.5% by weight of water, based on said formulation, which comprises
(A) to prepare the granules a), suspending or, at a temperature of not higher than 95° C., dissolving, a film-forming and water-soluble polymer and suspending a microorganism in this suspension or solution after cooling to room temperature,
(B) to prepare the granules b), suspending a carboxyl group-containing or sulfate group-containing polysaccharide in an aqueous buffer solution containing potassium ions, and then suspending the microorganism in this solution,
(C) spraying the resultant suspensions direct on to a finely particulate substrate or mixing said suspensions with the finely particulate substrate, and
(D) removing the water to a concentration which is not less than 0.5% by weight, based on the granular formulation.

36. A process according to claim 35, wherein, if a suspension of a film-forming and water-soluble polymer is used for the preparation of granular formulation a), said suspension is preferably prepared in the temperature range from 10° to 30° C.

37. A process according to claim 35, wherein, if a solution of a film-forming and water-soluble polymer is used for the preparation of granular formulation a), said solution is preferably prepared in the temperature range from 25° to 95° C.

38. A process according to claim 35, wherein the microorganism is added at a temperature of less than 40° C. to the solution or suspension of the polymer.

39. A process according to claim 35, wherein the microorganism is added at a temperature of less than 30° C.

40. A process according to claim 35, wherein the buffer is a mixture of potassium hydrogen phosphate and potassium monohydrogen phosphate.

41. A process according to claim 40, wherein the pH of the solution or suspension is 7.

42. A process according to claim 39, wherein the buffer concentration is from 0.00001 M/I to 1 M/l.

43. Use of the granular formulation for protecting plants from attack by disease or damage by insects.
